(19) United States
(12) Patent Application Publication
(10) Pub. No.: US 2010/0143877 A1
(43) Pub. Date: Jun. 10, 2010

(54) Composition for preserving reproductive cells and method of using

(75) Inventor: Kevin Rozeboom, Mt. Horeb, WI (US)

Correspondence Address: BOYLE FREDRICKSON S.C., 840 North Plankinton Avenue, MILWAUKEE, WI, 53203, US

(73) Assignee: ReproQuest, LLC

(21) Appl. No.: 11/515,570

(22) Filed: Sep. 5, 2006

(51) Int. Cl. A01N 1/02; C12N 5/06

(52) U.S. Cl. 435/2; 435/402

(57) ABSTRACT

Disclosed are compositions for mammalian, avian or piscian reproductive cells and methods for the collection, holding, processing, in vitro fertilization, sexing culturing, or storing (including long-term cryopreservation) of mammalian or avian reproductive sperm cells. The compositions comprise suitable reproductive cell media and antioxidant bioflavonoids. These may be comprised of Oligomeric Proanthocyanidins (OPC) which include the specific molecules of OPCs, that are namely; (epicatechin (EC), epigallocatechin (EGC), epicatechin gallate (ECG), and epigallocatechin gallate (EGCG)).

### FIELD OF THE INVENTION

The present invention relates to extender compositions for the preservation of animal cells. More specifically, the present invention relates to extender compositions comprising antioxidants for the preservation of sperm cells and other reproductive media for use in artificial insemination.

### BACKGROUND OF THE INVENTION

Artificial insemination (AI), along with in vitro fertilization and embryo transplantation, afford enhanced reproduction in animals, including livestock, and offer many advantages over direct mating. In the livestock breeding art, these techniques permit wider dissemination of desirable genetic features. Semen collected from a single male can be used to inseminate multiple females, thereby reducing the number of males required to maintain a population. AI techniques permit greater control over breeding, which results in greater reproducibility and facilitates maintenance of large-scale operations.

Maintaining the viability of reproductive cells is an important aspect of artificial insemination and other techniques used in indirect breeding. The processing requirements for semen used in AI may vary according to the species of animal. For example, bovine insemination requires relatively low concentrations of semen, and a suitable sample may be rapidly frozen in a narrow diameter straw and stored for an extended period of time without adversely affecting the fertility of the sample. In contrast, for example, porcine semen is not suitable for this approach because greater numbers of sperm cells and larger volumes of semen or diluted semen are required to inseminate sows. Insemination using frozen boar semen has not been sufficiently satisfactory to justify widespread use of AI. Boar semen is generally diluted or extended with a suitable storage medium and cooled to a temperature of about 17 degrees Celsius prior to transport. The storage medium serves to increase the total volume of the sample and provide nutrients to maintain the sperm cells. Significant loss of sperm cell vitality occurs after storing the semen for just a few days.

Currently, the best media generally maintain boar sperm cell viability for about three to seven days. This relatively short storage time imposes considerable constraints on the distribution of boar semen for use in AI. Other animals, such as horses, produce sperm cells that also suffer from short-lived viability. Artificial insemination, in vitro fertilization, and embryo transfer technology are also used in humans to aid in the conception process, and/or as a solution to various physiological problems relating to infertility. Clearly, maintaining the viability of reproductive cells for these uses is also very important.

Many compositions for preserving semen are currently commercially available, including short-term, medium-term, and long-term extenders. Typically, storage medium formulations are provided in solid form and are diluted with water for use. Standard formulations (e.g., Androhep, Modena, and BTS) can be found in the art. For example, see Waberski, et al., “Fertility of long-term-stored boar semen: Influence of extender (Androhep and Kiev) storage time and plasma droplets of the semen”, Anim. Reprod. Sci. 36:145-151 or Levis, “Liquid Boar Semen Production: Current Extender Technology and Where do we go from here”, Boar Semen Preservation IV, (2000). Ed. L. A. Johnson and H. D. Guthrie, Allen Press, Inc., which are incorporated herein in their entirety.

Reproductive cell media generally contain physiologically balanced salts, energy sources, and antibiotics and are suitable for the species whose reproductive cells are being treated. Typically, suitable media contain at least one buffer (e.g., sodium bicarbonate or HEPES) and a carbon source (e.g., glucose). Additional components may include ethylene diamine tetraacetic acid (EDTA), bovine serum albumin (BSA), and one or more antibiotics. Examples of suitable media for species such as humans and monkeys include: human tubal fluid (HTF), as obtained from Quinn et al., Fertil. Steril., 44: 493 (1985), supplemented with 10% heat-inactivated maternal or fetal cord serum, which is typically used for IVF and embryo culture; TALP, as obtained from Boatman, in In Vitro Growth of Non-Human Primate Pre- and Peri-implantation Embryos, ed. Bavister, pp. 273-308 (New York: Plenum Press, 1987); Ham's F-10 medium, Menezo's B.sub.2 medium (BioMerieux SA, France), Earles medium (Sigma Chemical Co., St. Louis, Mo.), and the like. General discussion describing these types of media are included in Menezo and Khatchadourian, “The Laboratory Culture Media,” Assisted Reproduction Reviews, 1: 136 (1991) and Lease, “Metabolism of the Preimplantation Mammalian Embryo,” Oxford Reviews of Reproductive Biology, 13: 35-72 (1991), ed. S. R. Milligan, Oxford University Press. The practitioner will be able to devise the necessary medium suitable for the species and the reproductive cell type. The pH of the medium is generally about 6.5 to 7.5 and preferably about 6.8-7.2.

The aging of living organisms is due to cross-linking of cellular proteins as well as strands of DNA and RNA, which control the rate of aging. This cross-linking occurs as a result of free radical activity. This theory has been confirmed as one of the major causes of aging. Free radicals are unstable forms of oxygen that occur within the body from normal metabolism, the digestion of dietary fat, and from exposure to certain chemicals, environmental pollutants, sunlight, radiation, burns, cigarette smoke, drugs, alcohol, viruses, bacteria, and parasites. This free radical oxidation occurs throughout the body, destroying cell membranes and cellular components as well as collagen and elastin.

Oligomeric Proanthocyanidins (OPCs) derived commercially from grapes and pine trees, are a mixture of antioxidant molecules, variously called proanthocyanidins, procyanidins, proanthocyanidolic oligomers (PCO) or oligomeric proanthocyanidins.

OPCs are a set of bioflavonoid complexes that perform as free radical scavengers in the human body. Many names refer to this set of bioflavonoids, including Oligomeric Procyanidolic Complexes, leucoanthocyanin, anthocyanidin and many others. OPCs are found in many plants throughout the plant kingdom with varying degrees of concentration. As mentioned, most notably Proanthocyanidins are found in pine bark, grape seed, and grape skin. However, bilberry, cranberry, black currant, green tea, black tea, and other plants also contain these flavonoids.

OPCs are a complex of specific molecules, technically known as a flavan-3-ol molecule (also known as a catechin). It is extremely unique that certain plants can bond flavan-3-ol molecules to form entirely new oligomeric molecular configurations. Two flavan-3-ol molecules together form a “dimer,” and three molecules bonded together form a “trimer.”

By itself, the flavan-3-ol molecule is not highly bioavailable and has less biological activity than OPC in the body. However, bonded together as dimers and trimers the flavan-3-ol molecules become extremely biologically active in a profoundly effective way in the human body. As a result, they are the source of a stunning array of proven health benefits.

Traditionally pine bark and grape seed have been used as sources of OPCs. These substances contain substantial amounts of four chemically similar molecules that have varying degrees of antioxidant ability. The most basic form, and least potent antioxidant, is epicatechin (EC). Additionally, epigallocatechin (EGC), epicatechin gallate (ECG), and epigallocatechin gallate (EGCG), which are more potent antioxidants, are also found therein. Green tea is a common source of EGCG, as it contains greater amounts of EGCG than both pine bark and grape seed. The content of EGCG in grape seed is about 15 percent of the total OPCs present. In green tea extracts, the amount of EGCG is 50 percent of the total OPCs present.

OPCs are useful for treating various diseases and have numerous uses in other biological activities. For example, OPCs are useful in treating vascular diseases because they actually increase the structural strength of weakened blood vessels. OPCs are one of the most potent antioxidants known—fifty times as powerful as vitamin E, according to some tests. OPCs can help neutralize the underlying chemical cause (free radicals) that promotes many diseases.

### BRIEF SUMMARY OF THE INVENTION

In one aspect, the present invention provides a composition comprising a reproductive cell medium for mammalian or avian reproductive cells, wherein the medium comprises at least one bioflavonoid complex selected from the following group: Oligomeric Procyanidolic Complexes (PCOs), leucoanthocyanin, and anthocyanidin. Sources for the extracts include: pine bark, grape seed, grape skin, bilberry, cranberry, black currant, green tea, black tea, and other plants.

In another aspect, the present invention provides a composition comprising a reproductive cell medium for mammalian or avian reproductive cells, wherein the medium comprises at least one OPC molecule, namely epicatechin (EC). Alternatively, the medium may comprise one of epigallocatechin (EGC), epicatechin gallate (ECG), and epigallocatechin gallate (EGCG).

Various alternative embodiments and modifications to the invention will be made apparent to one of ordinary skill in the art by the following detailed description taken together with the drawings.

### DETAILED DESCRIPTION

The present invention includes compositions comprising media for reproductive cells. In one embodiment, the invention provides compositions comprising sperm cell media for mammalian or avian sperm cells. As used herein, the term “reproductive cells” encompasses not only sperm cells, but also, oocytes, and embryos of any animal, including livestock (e.g., pigs, cows, horses, sheep, and the like) and humans. Further, the terms “medium for reproductive cells”, or “reproductive cell medium” refer to any medium used for the collection, holding, processing, in vitro fertilization, sexing, culturing, or storing (including long-term cryopreservation) of reproductive cells and includes both solid and liquid compositions, as well as solid compositions that are reconstituted or mixed with a liquid carrier, such as water, for use. The term “sperm cell medium” refers to any medium used for the collection, holding, processing, in vitro fertilization, sexing, culturing, or storing (including long-term cryopreservation) of sperm cells and/or semen.

In a first aspect, the present invention provides a composition comprising a reproductive cell medium for mammalian or avian reproductive cells, particularly a sperm cell medium for mammalian or avian sperm cells, where the medium comprises at least one OPC selected from the group consisting of extracts of pine bark, grape seed and grape skin, bilberry, cranberry, black currant, green tea, black tea, and other plants and preferably all of the aforementioned extracts are present in the media of the present invention. These OPCs may be obtained from any commercially available source.

The concentration of OPCs present in the medium may be in any amount desired by the medium formulator. The amounts below are expressed as the concentration of a given OPC in a composition when the medium is in the liquid state upon reconstitution, dilution, or mixing with water or other suitable carriers. As stated above, each OPC may be used alone or in combination with one or more other OPCs. More specifically, grape seed, red wine, pine bark, bilberry extract, green tea extract, citrus bioflavonoid extract may be present in a concentration of from about 0.1 ng/L to about 3 g/L.

Preferably, the chosen extracts are present in substantially equal concentrations ranging from about 10 mg/L to 300 mg/L. More preferably, the extracts are present in concentrations ranging from about 50 mg/L to 130 mg/L. Of course, the concentration of OPCs present in a given medium depends on several factors including the purity of the extract and the type of reproductive cells (e.g., mammalian, avian, piscean) with which the medium is to be used.

The optimal concentration of OPCs to be used for a given medium can also be determined by preparing a series of media with differing concentrations of OPCs and comparing the efficacy of those media for use with a given type of reproductive cell. For example, where porcine semen is used, efficacy can be determined by using one or more known measurements of sperm viability, including both in vitro and in vivo techniques. One indicia of sperm cell viability is motility. Increased motility of sperm cells stored in a supplemental storage medium relative to the motility of sperm cells stored in an unsupplemented storage medium is indicative of enhanced viability. Enhanced viability of cryopreserved sperm cells is also suggested by comparatively lower percentages of acrosome damaged sperm, increased percentage of membrane intact spermatozoa, increased survivability of sperm following cryopreservation, and increased pregnancy rates or litter size following artificial insemination using stored semen. Similarly, enhanced viability of sperm, oocytes, or embryos can be indicated by increased pregnancy rates or litter size following in vitro fertilization or embryo transfer.

In a second aspect of the present invention, the composition comprising a reproductive cell medium for reproductive cells, wherein the medium comprises EGCGs, is provided. EGCGs may be used in similar concentrations as OPCs.

For example, to obtain a certain volume of medium, the appropriate mass of the individual components needed to obtain the desired final concentration may be combined with water or other suitable solvent and brought to the desired final volume. The media may be conveniently prepared as a solid, blended formulation in which each of the individual components, including OPCs or EGCGs, are added in dry form and the components blended together for later reconstitution to give the desired final concentration of each component. A suitable commercially available dry medium (e.g., _Preserv Xtra) and the desired amount of OPCs or EGCGs, for example, could be reconstituted in water to obtain a medium with the desired final concentration of components. It is envisioned that concentrated stock solutions of the media of the invention may be prepared and subsequently diluted to achieve the appropriate final concentration of components in the medium prior to use.

The concentrations of components expressed herein are given as the final concentration of components in the medium for reproductive cells. One skilled in the art would appreciate that the dry blended formula is formulated such that the masses of each individual media component are present in an amount sufficient to give the desired concentration when the blended formula is reconstituted with a suitable volume of water. The concentration of each component (e.g., grape seed extract, red wine extract, pine bark extract, bilberry extract, green tea extract or citrus bioflavonoids extract) may also be expressed in terms of the units of mass of each component of interest per unit of mass of the dry blended media. It will be appreciated by one skilled in the art that concentrations expressed on a weight-by-weight basis may vary depending on the mass contribution of other components in the medium. Concentrations of components in a liquid medium are expressed in terms of units of mass per liter. One of skill in the art would appreciate that the medium of the present invention may be prepared in any volume, and the invention is not intended to be limited to media prepared in one-liter volumes.

In another aspect of the present invention, a method of storing mammalian or avian reproductive cells comprising contacting the cells with the compositions of the present invention is provided. Typically, as stated above, the compositions of the invention will be provided in solid form. It should then be diluted with Type I or Type II water, approximately one hour prior to use. Preferably, a sample comprising the cells is collected by any suitable means and placed in contact with a given amount of composition in liquid form as soon as possible following collection. The contacting step should be performed in such a manner that mechanical or other injury to the cells is minimized. Following the contacting step, the mixture of cells and composition are preferably equilibrated to and held at a suitable temperature for maintaining the viability of the cells until use. The temperature at which the cells are suitably maintained will depend on the type of cell, medium and application.

The following non-limiting examples are intended to be purely illustrative. In the examples below, commercially available semen extenders were combined with grape seed extract, red wine extract, pine bark extract, bilberry extract, green tea extract or citrus extract bioflavonoids and water to prepare compositions comprising sperm cell media according to the present invention. The compositions were evaluated for their ability to enhance or extend the viability of stored boar semen under conditions of enhanced metabolic activity that produces a state of increased free radical production.

### Treatment Preparation

Compositions comprising sperm cell media were prepared prior to collection by reconstituting in BTS, or otherwise commonly known as Beltsville Thawing Solution from Pursel and Johnson et al., 1975 (Pursel, V. G. and L. A. Johnson. 1975. Freezing of boar spermatozoa; Fertilizing capacity with concentrated semen and a new thawing procedure. J. Anim. Sci. 42: 927-931), and the individual components indicated in the experiments below with microfiltered deionized (Type I) water. BTS was prepared according to the manufacturer's instructions, except that additional components were added in an amount sufficient to give the concentrations indicated below. Each composition was transferred in 75-ml aliquots to 100 -ml plastic bottles commercially available from Swine Genetics International, Cambridge, Iowa.

### Semen Collection, Processing, and Storage

Semen was collected (modified full ejaculate) from randomly selected, sexually mature boars using the gloved-hand technique. Following each collection, each ejaculate was evaluated for sperm cell concentration with a photometer having a 546-nanometer filter. The percentage of motile cells was assessed by estimating the number of moving cells in groups of ten (10) cells and by counting at least ten (10) groups of cells. Aliquots of semen containing 1×10⁹ motile spermatozoa from each of the three boars were transferred into each 75 milliliter aliquot of sperm cell media at the same temperature (36° C. +/− 0.1° C.) to give a final concentration of 4×10⁷ live sperm cells per milliliter. Following dispersion of the sperm cells in the composition, each sample was tested for percent motility.

### Maintenance of Samples and Data Collection

Samples were maintained in a semen storage unit at 35° C. The samples were gently mixed periodically during storage. At days 0, 1 and 2, the samples were mixed and five-milliliter aliquots were removed for testing. Motility testing was performed on a plain glass slide pre-warmed to 37° C. The ability of OPCs to enhance sperm viability in stored semen was assessed by evaluating the motility of the sperm in semen stored in conditions promoting high metabolic activity and the production of free radicals. The resulting data was then compared to sperm cell motility in media not containing OPCs.

### EXAMPLE 1

Sperm cell media were prepared by combining BTS and grape seed extract, red wine extract, pine bark extract, bilberry extract, green tea extract or citrus extract bioflavonoids to give final concentrations of 8 mg/L, 16 mg/L or 25 mg/L of each of the extracts. Sperm motility was assessed at days 1 and 2 while being stored at 37° C. The data is summarized in Table 1 in terms of mean percent motility based on the assessment of twenty-seven samples for each medium tested. Media treated with OPCs in Boar B had greater motility under the kind of high metabolic conditions that produce greater levels of free radicals.

TABLE 1

| | | | Boar A Day | Boar A Day | | Boar B Day | Boar B Day | Mean % |
|---|---|---|---|---|---|---|---|---|
| Treatment | pH | msOm | 1 Motility % | 2 Motility % | Mean % | 1 Motility % | 2 Motility % | Motility |
| Control: | 7.41 | 340 | 94 | 89 | 91.5 | 90 | 70 | 80.0 |
| BTS 0 mg | | | | | | | | |
| OPC | | | | | | | | |
| 8 mg | 7.21 | 325 | 95 | 74 | 85.0 | 90 | 90 | 90.0 |
| OPC/liter | | | | | | | | |
| 16 mg | 7.03 | 323 | 96 | 72 | 84.0 | 95 | 79 | 87.0 |
| OPC/liter | | | | | | | | |
| 25 mg OPC/ | 6.76 | 320 | 93 | 49 | 71.0 | 90 | 43 | 66.5 |
| liter | | | | | | | | |

### EXAMPLE 2

Sperm cell media were prepared by combining BTS and an OPC complex of grape seed extract, red wine extract, pine bark extract, bilberry extract, green tea extract and citrus extract bioflavonoids to give final concentrations of 0 mg/L (Control), 60 mg/L, 130 mg/L, 250 mg/L of each of the complex. Sperm motility was assessed at 0, 7, 15, 19, 28, and 37 hours while being stored at 37° C. Means over time are pooled. The data is summarized in Table 2 in terms of mean percent motility, based on assessment of twenty-seven samples for each medium tested. Media treated with OPCs had significantly better motility under the kind of high metabolic conditions that produce greater levels of free radicals.

TABLE 2

| Trt | Motility | SE |
|---|---|---|
| +Control* | 48.13^{a} | 7.83 |
| Control** | 57.37^{ab} | 7.01 |
| 0.06 | 58.91^{b} | 6.14 |
| 0.13 | 58.06^{ab} | 6.12 |
| 0.25 | 50.83^{ab} | 6.52 |

^{abc}Columns with different superscripts are statistically different (P < 0.05).
*Treatment included EDTA in BTS formulation.
**Treatment did not include EDTA in BTS formulation.

### EXAMPLE 3

In another study, sperm cell media were prepared for cryopreservation using Westendorf medium (11% lactose, 25% egg yolk) and optimal concentrations of grape seed extract, red wine extract, pine bark extract, bilberry extract, green tea extract or citrus extract bioflavonoids (16.0 mg/L of each respectively). Freshly collected semen from 3 boars was pooled together, and the samples were transferred to aliquots of media and centrifuged. Semen was then prepared for cryopreservation with or without OPCs (treatment vs. control, respectively) Motility was assessed following cryopreservation using liquid nitrogen and then thawing at 50° C. for 15 seconds. The data is summarized in Table 3 below in terms of mean percent survivability and percent normal acrosomes based on the assessment of 116 and 598 samples for each medium tested. Media treated with OPCs had significantly better survival and membrane integrity following cryopreservation.

TABLE 3

| | | Post thaw | Standard | P Value vs | | Normal | Standard | P Value vs |
|---|---|---|---|---|---|---|---|---|
| Treatment | N | survival % | error | control | N | Acrosome % | Error | control |
| Control | 116 | 76.0 | 2.2 | | | 76.0 | 2.2 | |
| 16 mg/liter | 116 | 88.0 | 2.2 | <.0001 | | 87.7 | 2.2 | <.0001 |

Various alternatives are contemplated as being within the scope of the following claims particularly pointing out and distinctly claiming the subject matter regarded as the invention.

1. A biological culture medium useful for animal reproduction comprising:
a semen culture medium including,
a) a semen sample; and
b) a volume of media having multiple dried components that are blended together and diluted before being combined with the semen sample, at least one of the multiple dried components being an oligomeric proanthocyanidin (OPC),
wherein the OPC mitigates degradation of the semen sample without detrimentally affecting a motility value of the semen sample when used in the semen culture medium, and wherein the semen culture medium is stored at a cooled temperature defined within a range of storage temperatures that prevents freezing of the semen culture medium.

2-9. (canceled)

10. The biological culture medium as recited in claim 1, wherein the volume of media containing an OPC includes at least one of leucoanthocyanin and anthocyanidin.

11. The biological culture medium as recited in claim 1, wherein the OPC is derived from one of pine bark, grape seed, grape skin, bilberry, cranberry, black currant, green tea, and black tea.

12-21. (canceled)

22. The biological culture medium as recited in claim 1, wherein the multiple dried components are reconstituted with purified water and then diluted before being combined with the semen sample.

23. A method of preparing a volume of extended porcine boar semen, for non-frozen temporary storage, comprising:
a) providing a powdered media having,
i) a first powdered component; and
ii) a second powdered component blended, wherein at least one of the first and second powdered components is an oligomeric proanthocyanidin (OPC);
b) blending the first and second powdered components with each other;
c) creating a solution by diluting the blended first and second powdered components with purified water;
d) adding a collected sample of porcine boar semen to the solution, promptly after collection thereof; and
e) equilibrating the combined sample and solution and defining a volume of extended porcine boar semen,
wherein the OPC mitigates degradation of the semen sample without detrimentally affecting a motility value of the semen sample and wherein the solution has a pH value of less than about 7.5 and an osmolarity of less than about 350 mOsm.

24. The method as recited in claim 23, wherein at least two different OPCs are provided in the powdered media.

25. The method as recited in claim 24, wherein at least three different OPCs are provided in the powdered media.

26. The method as recited in claim 25, wherein at least four different OPCs are provided in the powdered media.
